(19) United States
(12) Patent Application Publication
(10) Pub. No.: US 2025/0011882 A1
(43) Pub. Date: Jan. 9, 2025

(54) Compositions and Methods for Detecting vanA and/or vanB Genes Associated with Multidrug Resistance

(71) Applicant: Roche Molecular Systems, Inc., Pleasanton, CA (US)

(72) Inventors: Kyle C. Cady, Redwood City, CA (US); Ryan Chan, San Jose, CA (US); Sharon Hochen Chiu, Sunnyvale, CA (US); Patrick Lin, San Jose, CA (US); Arrash Moghaddasi, Portola Valley, CA (US); Eugene Spier, Los Altos, CA (US); Jingtao Sun, San Ramon, CA (US)

(21) Appl. No.: 18/711,732

(22) PCT Filed: Nov. 21, 2022
(86) PCT No.: PCT/EP2022/082684

§ 371 Date: May 20, 2024

Related U.S. Application Data
(60) Provisional application No. 63/264,380, filed on Nov. 22, 2021.

(51) Int. Cl. C12Q 1/689; C12Q 1/6851

(52) U.S. Cl. CPC C12Q 1/689; C12Q 1/6851; C12Q 2600/16

(57) ABSTRACT

Methods for the rapid detection of the presence or absence of bacteria having the vanA and/or vanB resistance mechanisms in a biological or non-biological sample are described. The methods can include performing an amplifying step, a hybridizing step, and a detecting step. Furthermore, primers, probes targeting the genes for vanA and vanB, along with kits are provided that are designed for the detection of vanA and vanB.

### FIELD OF THE INVENTION

The present disclosure relates to the field of bacterial diagnostics, and more particularly to detection of vancomycin-resistant bacteria such as Staphylococcus aureus that contain vanA and/or vanB nucleic acid sequences.

### BACKGROUND OF THE INVENTION

Vancomycin is a glycopeptide antibiotic that has strong activity against many Gram-positive organisms. In many treatment regimens, it is considered a “drug of last resort” (DoLR), meaning it is only used after all other drug options have failed. Because it is a DoLR, it is critical to identify whether a patient is colonized or infected with bacteria that are resistant to vancomycin.

The van gene is a resistance mechanism that confers resistance to Vancomycin by altering peptidoglycan synthesis, and its presence has been documented across many Gram-positive species. Multiple variants/types of the gene exist—vanA/B/C/DE/G—each possessing unique characteristics in terms of resistance profile, transmissibility, and prevalence. In particular, the vanA and vanB genes are of interest, because they can exist on transposons (i.e. the resistance gene can be transferred), and because of their prevalence worldwide. Examples of clinically relevant vancomycin resistant bacteria include Vancomycin Resistant Enterococcus (VRE) and Vancomycin Resistant Staphylococcus aureus (VRSA).

The vanA genes encode proteins that confer high-level resistance to vancomycin and teicoplanin (Arthur et al., 1993, 1996). The expression of vanA genes is induced by either vancomycin or teicoplanin. VanA-type glycopeptide resistance has been described for several enterococcal species (see Mendez-Alvarez et al., 2000 for review). The vanB genes confer resistance to various concentrations of vancomycin but not teicoplanin (Baptista et al., 1996; Evers & Courvalin, 1996), and are induced only by vancomycin and not by teicoplanin. VanB-type glycopeptide resistance has been described for Enterococcus faecalis and Enterococcus faecium. Recently the genomes of some methicillin-resistant Staphylococcus aureus (MRSA) strains evolved as a result of the acquisition of the vanA gene from enterococci to generate vancomycin-resistant S. aureus (VRSA) strains. Thus, there is a need in the art for a quick and reliable method to specifically detect both vanA- and vanB-containing bacteria in a specific and sensitive manner.

### SUMMARY OF THE INVENTION

Certain aspects of the present invention relate to methods for the rapid detection of the presence or absence of bacteria having the vanA and/or vanB resistance mechanisms in a biological or non-biological sample, for example, multiplex detection of vanA and vanB by real-time polymerase chain reaction in a single test tube. Embodiments include methods of detection of the vanA and vanB resistance mechanisms by determining the presence of a vanA gene and/or vanB gene comprising performing at least one cycling step, which may include an amplifying step and a hybridizing step. Furthermore, embodiments include primers, probes, and kits that are designed for the detection of the vanA and vanB resistance mechanisms in a single tube. The detection methods are designed to target the vanA gene and the vanB gene, which allows one to detect vancomycin-resistant bacterial strains in a single test. In one embodiment, the vancomycin-resistant bacterial strain is Staphylococcus aureus.

In one aspect, a method for detecting bacteria having a vanA gene and/or vanB gene in a sample is provided, including performing an amplifying step including contacting the sample with a set of vanA forward and reverse primers and a set of vanB forward and reverse primers to produce an amplification product if the vanA and/or van gene is present in the sample; performing a hybridizing step including contacting the amplification product with one or more detectable vanA probes and one or more detectable vanB probes; and detecting the presence or absence of the amplification product, wherein the presence of the amplified product is indicative of the presence of the vanA and/or vanB gene in the sample and wherein the absence of the amplified product is indicative of the absence of the vanA and/or vanB gene in the sample; wherein the set of vanA primers comprises or consists of a forward primer comprising or consisting of an oligonucleotide sequence of SEQ ID NOs: 1 or 2, or a complement thereof, and a reverse primer comprising or consisting of an oligonucleotide sequence of SEQ ID NOs: 3 or 4, or a complement thereof; the set of vanB primers comprises or consists of a forward primer comprising or consisting of an oligonucleotide sequence selected from the group consisting of SEQ ID NOs: 6, 7, 11 and 12, or a complement thereof and a reverse primer comprising or consisting of an oligonucleotide sequence selected from the group consisting of SEQ ID NOs: 8, 9, 13 and 14, or a complement thereof; and wherein the one or more detectable vanA probe comprises or consists of the oligonucleotide sequence of SEQ ID NO: 5, or a complement thereof, and the one or more detectable vanB probe comprises or consists of the oligonucleotide sequence of SEQ ID NOs: 10 or 15, or a complement thereof. In some embodiments, the hybridizing step comprises contacting the amplification product with a detectable probe that is labeled with a donor fluorescent moiety and a corresponding acceptor fluorescent moiety; and the detecting step comprises detecting the presence or absence of fluorescence resonance energy transfer (FRET) between the donor fluorescent moiety and the acceptor fluorescent moiety of the probe, wherein the presence or absence of fluorescence FRET is indicative of the presence or absence of in the sample. In some embodiments, the amplifying step employs a polymerase enzyme having 5′ to 3′ nuclease activity. In some embodiments, the donor fluorescent moiety and the corresponding acceptor fluorescent moiety are within no more than 8 nucleotides of each other on the probe. In certain embodiments, the acceptor fluorescent moiety is a quencher. In one embodiment, the set of vanA primers comprises a forward primer comprising an oligonucleotide sequence of SEQ ID NO: 2, and a reverse primer comprising an oligonucleotide sequence of SEQ ID NO: 4; and the set of vanB primers comprises a forward primer comprising an oligonucleotide sequence of SEQ ID NO: 7 and a reverse primer comprising an oligonucleotide sequence of SEQ ID NO: 9; the one or more detectable vanA probe comprises an oligonucleotide sequence of SEQ ID NO: 5 and the one or more detectable vanB probe comprises an oligonucleotide sequence of SEQ ID NO: 10.

In another aspect, the present invention relates to a primer and probe set for amplification and detection of vanA gene target sequence comprising or consisting of (at least) one forward primer comprising or consisting of an oligonucleotide sequence selected from SEQ ID NOs: 1 or 2, or a complement thereof; (at least) one reverse primer comprising or consisting of an oligonucleotide sequence selected from SEQ ID NOs: 3 or 4, or a complement thereof, and (at least) one detectable probe for detection of an vanA amplification product comprising or consisting of an oligonucleotide sequence of SEQ ID NO: 5, or a complement thereof. In one embodiment, the detectable probe comprises a donor fluorescent moiety and a corresponding acceptor fluorescent moiety. In certain embodiments, the acceptor fluorescent moiety is a quencher.

In another aspect, the present invention relates to a primer and probe set for amplification and detection of vanB gene target sequence comprising or consisting of (at least) one forward primer comprising or consisting of an oligonucleotide sequence selected from SEQ ID NOs: 6, 7, 11 and 12, or a complement thereof, (at least) one reverse primer comprising or consisting of an oligonucleotide sequence selected from SEQ ID NOs: 8, 9, 13 and 14, or a complement thereof, and (at least) one detectable probe for detection of vanB amplification product comprising or consisting of an oligonucleotide sequence selected from SEQ ID NOs: 10 and 15, or a complement thereof. In one embodiment, the detectable probe comprises a donor fluorescent moiety and a corresponding acceptor fluorescent moiety. In certain embodiments, the acceptor fluorescent moiety is a quencher. In another aspect, the present invention relates to a primer set for amplification of the vanA gene target comprising at least one primer comprising or consisting of an oligonucleotide sequence selected from SEQ ID NOs: 1, 2, 3, and 4, or a complement thereof, and a detectable probe for detection of the vanA amplification product comprising or consisting of an oligonucleotide sequence of SEQ ID NO: 5, or a complement thereof; and/or a primer set for amplification of the vanB gene target comprising at least one primer comprising or consisting of an oligonucleotide sequence selected from SEQ ID NOs: 6, 7, 8, 9, 11, 12, 13 and 14 or a complement thereof, and a detectable probe for detection of the vanB amplification product comprising or consisting of an oligonucleotide sequence selected from SEQ ID NOs: 10 and 15, or a complement thereof. In one embodiment, the detectable probe comprises a donor fluorescent moiety and a corresponding acceptor fluorescent moiety. In certain embodiments, the acceptor fluorescent moiety is a quencher.

In still another aspect, the present invention provides an oligonucleotide comprising or consisting of a sequence of nucleotides selected from SEQ ID NOs: 1-15, or a complement thereof, which oligonucleotide has 100 or fewer nucleotides. In another embodiment, the present disclosure provides an oligonucleotide that includes a nucleic acid having at least 70% sequence identity (e.g., at least 75%, 80%, 85%, 90% or 95%, etc.) to one of SEQ ID NOs: 1-15, or a complement thereof, which oligonucleotide has 100 or fewer nucleotides. Generally, these oligonucleotides may be primer nucleic acids, probe nucleic acids, or the like in these embodiments. In certain of these embodiments, the oligonucleotides have 40 or fewer nucleotides (e.g. 35 or fewer nucleotides, 30 or fewer nucleotides, etc.) In some embodiments, the oligonucleotides comprise at least one modified nucleotide, e.g. to alter nucleic acid hybridization stability relative to unmodified nucleotides. Optionally, the oligonucleotides comprise at least one label and/or at least one quencher moiety. In some embodiments, the oligonucleotides include at least one conservatively modified variation. “Conservatively modified variations” or, simply, “conservative variations” of a particular nucleic acid sequence refers to those nucleic acids, which encode identical or essentially identical amino acid sequences, or, where the nucleic acid does not encode an amino acid sequence, to essentially identical sequences. One of skill will recognize that individual substitutions, deletions or additions which alter, add or delete a single amino acid or a small percentage of amino acids (typically less than 5%, more typically less than 4%, 2% or 1%) in an encoded sequence are “conservatively modified variations” where the alterations result in the deletion of an amino acid, addition of an amino acid, or substitution of an amino acid with a chemically similar amino acid. In some embodiments, such oligonucleotides are suitable for being used in the methods, the primer and probe sets and the kits according to the invention.

In one embodiment, amplification can employ a polymerase enzyme having 5′ to 3′ nuclease activity. Thus, the first and second fluorescent moieties may be within no more than 8 nucleotides of each other along the length of the probe. In another aspect, the vanA and vanB probes includes a nucleic acid sequence that permits secondary structure formation. Such secondary structure formation generally results in spatial proximity between the first and second fluorescent moiety. According to this method, the second fluorescent moiety on the probe can be a quencher.

In a further aspect, the present invention provides for a kit for detecting one or more nucleic acids of vanA and/or vanB resistance mechanisms. The kit can include a plurality of sets of vanA and/or vanB primers specific for amplification of the vanA gene target and/or the vanB gene target; and one or more detectable vanA and/or vanB probes specific for detection of the vanA and/or vanB amplification products.

In one aspect, a kit for detecting a nucleic acid of vanA-containing Staphylococcus aureus is provided comprising a primer and probe set comprising or consisting of (at least) one forward primer comprising or consisting of an oligonucleotide sequence selected from SEQ ID NOs: 1 or 2, or a complement thereof, (at least) one reverse primer comprising or consisting of an oligonucleotide sequence selected from SEQ ID NOs: 3 or 4, or a complement thereof, and (at least) one detectable probe for detection of vanA amplification product comprising or consisting of an oligonucleotide sequence of SEQ ID NO: 5, or a complement thereof and further comprising at least one of nucleoside triphosphates, a nucleic acid polymerase, and a buffer necessary for the function of the nucleic acid polymerase.

In one aspect, a kit for detecting a nucleic acid of vanB-containing Staphylococcus aureus is provided comprising a primer and probe set comprising or consisting of (at least) one forward primer comprising or consisting of an oligonucleotide sequence selected from SEQ ID NOs: 6, 7, 11 and 12, or a complement thereof, (at least) one reverse primer comprising or consisting of an oligonucleotide sequence selected from SEQ ID NOs: 8, 9, 13 and 14, or a complement thereof, and (at least) one detectable probe for detection of van amplification product comprising or consisting of an oligonucleotide sequence selected from SEQ ID NOs: 10 and 15, or a complement thereof and further comprising at least one of nucleoside triphosphates, a nucleic acid polymerase, and a buffer necessary for the function of the nucleic acid polymerase.

In another aspect, a kit for detecting a nucleic acid of vanA- and/or vanB-containing Staphylococcus aureus comprising a primer and probe set for amplification and detection of a vanA nucleic acid and a primer and probe set for amplification and detection of a vanB nucleic acid and further comprising at least one of nucleoside triphosphates, a nucleic acid polymerase, and a buffer necessary for the function of the nucleic acid polymerase. Herein, the vanA primer and probe set may comprise or consist of (at least) one forward primer comprising or consisting of an oligonucleotide sequence selected from SEQ ID NOs: 1 or 2, or a complement thereof, (at least) one reverse primer comprising or consisting of an oligonucleotide sequence selected from SEQ ID NOs: 3 or 4, or a complement thereof, and (at least) one detectable probe for detection of vanA amplification product comprising or consisting of an oligonucleotide sequence of SEQ ID NO: 5, or a complement thereof and/or the vanB primer and probe set may comprise or consist of (at least) one forward primer comprising or consisting of an oligonucleotide sequence selected from SEQ ID NOs: 6, 7, 11 and 12, or a complement thereof, (at least) one reverse primer comprising or consisting of an oligonucleotide sequence selected from SEQ ID NOs: 8, 9, 13 and 14, or a complement thereof, and (at least) one detectable probe for detection of vanB amplification product comprising or consisting of an oligonucleotide sequence selected from SEQ ID NOs: 10 and 15, or a complement thereof.

In certain embodiments, the kits can include probes already labeled with donor and corresponding acceptor fluorescent moieties, or can include fluorophoric moieties for labeling the probes. The kit can also include nucleoside triphosphates, nucleic acid polymerase, and buffers necessary for the function of the nucleic acid polymerase either packaged in single tubes or combined in one tube. The kit can also include a package insert and instructions for using the primers, probes, and fluorophoric moieties to detect the presence or absence of the vanA gene and/or the vanB gene in a sample.

Another aspect of the present invention relates to methods for the rapid detection of Staphylococcus aureus (S. aures) having vancomycin resistance (VRSA), methicillin resistance (MRSA) or both VRSA and MRSA. The method comprises performing an amplifying step including contacting the sample with: a set of vanA forward and reverse primers and a set of vanB forward and reverse primers to produce an amplification product if the vanA gene and/or vanB gene is present in the sample; a set of forward and reverse primers to produce an amplification product of the S. aureus capsular polysaccharide enzyme (CPE) if S. aureus is present in the sample (as disclosed in U.S. Pat. No. 9,034,581 and incorporated by reference herein in its entirety); a set of forward and reverse primers to produce an amplification product of the right extremity junction of the Staphylococcal Chromosomal Cassette mec (SCCmec) mobile genetic element if SCCmec is present in the sample; and a set of mecA forward and reverse primers and a set of mecC forward and reverse primer to produce an amplification product if the mecA and/or mecC gene is present in the sample; performing a hybridizing step including contacting the amplification products with one or more detectable vanA probes, one or more detectable vanB probes, one or more detectable CPE probes, one or more detectable SCCmec probes, one or more detectable mecA probes, and one or more detectable mecC probes; and detecting the presence or absence of the amplification product, wherein the presence of the amplification product of the vanA gene and/or the vanB gene and the CPE gene is indicative of the presence of VRSA in the sample and wherein the presence of the amplification product of the SCCmec element, the mecA gene and/or the mecC gene and the CPE gene is indicative of the presence of MRSA in the sample. In one embodiment, the set of vanA forward and reverse primers for amplification of the vanA gene comprises at least one primer comprising or consisting of an oligonucleotide sequence selected from SEQ ID NOs: 1, 2, 3, and 4, or a complement thereof, and the one or more detectable probe for detection of the vanA amplification product comprises or consists of an oligonucleotide sequence of SEQ ID NO: 5, or a complement thereof. In one embodiment, the set of vanB forward and reverse primers for amplification of the vanB gene comprises at least one primer comprising or consisting of an oligonucleotide sequence selected from SEQ ID NOs: 6, 7, 8, 9, 11, 12, 13 and 14 or a complement thereof, and the one or more detectable probe for detection of the vanB amplification product comprises or consists of an oligonucleotide selected from SEQ ID NOs: 10 and 15, or a complement thereof. In one embodiment, the vanA primer and probe set may comprise or consist of (at least) one forward primer comprising or consisting of an oligonucleotide sequence selected from SEQ ID NOs: 1 or 2, or a complement thereof, (at least) one reverse primer comprising or consisting of an oligonucleotide sequence selected from SEQ ID NOs: 3 or 4, or a complement thereof, and (at least) one detectable probe for detection of vanA amplification product comprising or consisting of an oligonucleotide sequence of SEQ ID NO: 5, or a complement thereof and/or the vanß primer and probe set may comprise or consist of (at least) one forward primer comprising or consisting of an oligonucleotide sequence selected from SEQ ID NOs: 6, 7, 11 and 12, or a complement thereof, (at least) one reverse primer comprising or consisting of an oligonucleotide sequence selected from SEQ ID NOs: 8, 9, 13 and 14, or a complement thereof, and (at least) one detectable probe for detection of vanB amplification product comprising or consisting of an oligonucleotide sequence selected from SEQ ID NOs: 10 and 15, or a complement thereof. In particular embodiments, the set of vanA primers comprises a forward primer comprising an oligonucleotide sequence of SEQ ID NO: 2, and a reverse primer comprising an oligonucleotide sequence of SEQ ID NO: 4; and the set of vanB primers comprises a forward primer comprising an oligonucleotide sequence of SEQ ID NO: 7 and a reverse primer comprising an oligonucleotide sequence of SEQ ID NO: 9; the one or more detectable vanA probe comprises an oligonucleotide sequence of SEQ ID NO: 5 and the one or more detectable vanB probe comprises an oligonucleotide sequence of SEQ ID NO: 10. In some embodiments, the set of S. aureus CPE forward and reverse primers for amplification of the S. aureus CPE gene comprises at least one primer comprising or consisting of an oligonucleotide sequences selected from SEQ ID NOs: 16 and 17 or a complement thereof, and the one or more detectable probe for detection of the S. aureus CPE amplification product comprises or consists of an oligonucleotide sequence of SEQ ID NO: 18, or a complement thereof. In certain embodiments, the set of CPE primers comprises or consists of a forward primer comprising or consisting of an oligonucleotide sequence of SEQ ID NO: 16, or a complement thereof and a reverse primer comprising or consisting of an oligonucleotide sequence of SEQ ID NO: 17, or a complement thereof, and the one or more detectable CPE probe comprises or consists of an oligonucleotide sequence of SEQ ID NO: 18, or a complement thereof. In one embodiment, the set of SCCmec forward and reverse primers for amplification of the SCCmec element comprises at least one primer comprising or consisting of an oligonucleotide sequences selected from SEQ ID NOs: 19, 20, 21, 22, 23 and 24, or a complement thereof, and the one or more detectable probe for detection of the SCCmec amplification product comprises or consists of an oligonucleotide sequence of SEQ ID NOs: 25, or a complement thereof. In certain embodiments, the set of SCCmec primers comprises a forward primer comprising an oligonucleotide sequence selected from SEQ ID NOs: 19 and 20, or a complement thereof and a reverse primer comprising an oligonucleotide sequence selected from SEQ ID NOs: 21, 22, 23 and 24, or a complement thereof and the one or more detectable vanB probe comprises an oligonucleotide sequence of SEQ ID NO: 25, or a complement thereof. In one embodiment, the set of mecA forward and reverse primers for amplification of the mecA gene comprises at least one primer comprising or consisting of an oligonucleotide sequences selected from SEQ ID NOs: 26 and 27, or a complement thereof, and the one or more detectable probe for detection of the mecA amplification product comprises or consists of an oligonucleotide sequence of SEQ ID NO: 28, or a complement thereof. In certain embodiments, the set of mecA primers comprises a forward primer comprising an oligonucleotide sequence of SEQ ID NO: 26, or a complement thereof and a reverse primer comprising an oligonucleotide sequence of SEQ ID NO: 27, or a complement thereof, and the one or more detectable mecA probe comprises an oligo-nucleotide sequence of SEQ ID NO: 28, or a complement thereof. In one embodiment, the set of mecC forward and reverse primers for amplification of the mecC gene comprises at least one primer comprising or consisting of an oligonucleotide sequences selected from SEQ ID NOs: 29 and 30, or a complement thereof, and the one or more detectable probe for detection of the mecC amplification product comprises or consists of an oligonucleotide sequence of SEQ ID NO: 31, or a complement thereof. In certain embodiments, the set of mecC primers comprises a forward primer comprising an oligonucleotide sequence of SEQ ID NO: 29, or a complement thereof and a reverse primer comprising an oligonucleotide sequence of SEQ ID NO: 30, or a complement thereof, and the one or more detectable mecC probe comprises an oligonucleotide sequence of SEQ ID NO: 31, or a complement thereof.

Unless otherwise defined, all technical and scientific terms used herein have the same meaning as commonly understood by one of ordinary skill in the art to which this invention belongs. Although methods and materials similar or equivalent to those described herein can be used in the practice or testing of the present subject matter, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### BRIEF DESCRIPTION OF THE DRAWINGS

FIG. 1 shows the results of the PCR assay using non-benzylated vanA and vanB primers as described in Example 3.

FIG. 2 shows the results of the PCR assay using benzylated vanA and vanB primers as described in Example 3.

FIG. 3 shows the results of the multiplex PCR assay using sets of primers and probes that target vanA, vanB, CPE, SCCmec OrfX, mecA and mecC in various Staphylococcus aureus and Enterococcus strains

### DETAILED DESCRIPTION OF THE INVENTION

Diagnosis of bacteria having the vanA and/or vanB resistance mechanisms by nucleic acid amplification provides a method for rapidly and accurately detecting the bacterial infection. A real-time assay for detecting the vanA gene and the vanB gene in a sample is described herein. Primers and probes for detecting vanA and/or vanB are provided, as are articles of manufacture or kits containing such primers and probes. The increased sensitivity of real-time PCR for detection of vanA and/or vanB compared to other methods, as well as the improved features of real-time PCR including sample containment and real-time detection of the amplified product, make feasible the implementation of this technology for routine diagnosis of vanA and/or vanB infections (including but not limited to Vancomycin-resistant Enterococcus (VRE) and Vancomycin-resistant Staphylococcus aureus (VRSA)) in the clinical laboratory.

Glycopeptide resistance in Enterococci is phenotypically and genotypically heterogeneous. The genes responsible for inducible resistance to high levels of vancomycin and teicoplanin (VanA phenotype) are carried by the 10,851-bp Tn1546 transposon. Transposition of Tn1546 into self-transferable plasmids and subsequent transfer by conjugation appears to be responsible for the dissemination of this type of resistance. Nine polypeptides are encoded by Tn1546 that belong to five functional groups: transposition functions (ORF1 and ORF2), regulation of resistance gene expression (VanR and VanS), synthesis of depsipeptide d-Ala-d-lactate (VanH and VanA), hydrolysis of d-Ala-d-Ala-containing peptidoglycan precursors (VanX and VanY), and low-level teicoplanin resistance (VanZ). VanB-type resistance (various levels of resistance to vancomycin and susceptibility to teicoplanin) is also due to production of d-Ala-d-Lac. The VanB ligase of VanB-type strains is structurally and functionally similar to VanA. The vanB gene was found on composite transposon Tn1547, which, in turn, was part of larger conjugative chromosomally located elements (90 to 250 kb). In contrast to acquired VanA- and VanB-type resistance, VanC-type resistance (low level of resistance to vancomycin and susceptibility to teicoplanin) is an intrinsic property of motile enterococci. Resistance in these species is due to synthesis of dipeptide d-Ala-d-Ser by VanC ligases leading to production of cell wall precursors with reduced vancomycin affinity.

The methicillin-resistance gene mecA, and its homolog, mecC, both encode an altered methicillin-resistant penicillin-binding protein (PBP2a or PBP2′), a penicillin binding protein with reduced affinity for β-lactam rings (the primary active-site of the β-lactam antibiotics such as penicillins, cephalosporins and carbapenems) (Guignard et al., 2005, Curr Opin Pharmacol 5 (5): 479-89), that is not present in susceptible strains and is believed to have been acquired from a distantly related species. Both mecA and mecC is carried on a mobile genetic element, the Staphylococcal Chromosomal Cassette mec (SCCmec) of MRSA strains. SCC elements also occur in sensitive S. aureus but do not carry the mecA gene or the mecC gene or carry non-functional mecA or mecC gene. Such strains can be a source of false positive results, because they may have the same right extremity junction.

MRSA detection from nasal specimen by detecting the mecA gene or the mecC gene and a S. aureus specific gene will sometimes lead to low positive predictive values (PPV) due to the presence of varying amounts of both non-resistant S. aureus and methicillin-resistant coagulase-negative Staphylococci (MRCoNS). A combination of those is undistinguishable from MRSA, because of the presence of both targets. Depending on the prevalence of MRSA this situation leads up to 30% false positive results. For a better PPV, the chosen target needs to be unique for MRSA. The only target currently known is Staphylococcal Chromosomal Cassette (SCCmec), which amplifies the transposon integration

The detection of mecA/mecC-containing S. aureus (mecC-MRSA) utilizes a strategy to produce an amplicon at the RE junction between the S. aureus orfX gene and SCCmec carrying the mecA gene or the mecC gene which confers resistance to methicillin. To accomplish this, one primer is anchored in a highly conserved region of the orfX gene of S. aureus (orfX primer), and a second primer is located within the non-conserved RE junction of SCCmec (RE primer). The resulting amplicon from the two primers spans part of the orfX gene and part of SCCmec. Due to the non-homologous nature of SCCmec at the RE junction, several different RE primers are necessary in order to accomplish the most coverage of unique MRSA strains. This type of identification and detection of mecA/mecC-MRSA has been described by several groups, for example, in U.S. Pat. Nos. 7,449,289 and 7,838,221 by Huletsky et al; in U.S. Pat. No. 8,535,888 by Aichinger et al.; and in U.S. Pat. Nos. 9,920,381 and 10,190,178 by Johnson et al., each of which is hereby incorporated in its entirety by reference. However, as previously mentioned this strategy can be a source of false positive results if the mecA or mecC gene is missing (entirely or partially) or is non-functional.

As used herein, the term “amplifying” refers to the process of synthesizing nucleic acid molecules that are complementary to one or both strands of a template nucleic acid molecule (e.g., vanA and/or vanB). Amplifying a nucleic acid molecule typically includes denaturing the template nucleic acid, annealing primers to the template nucleic acid at a temperature that is below the melting temperatures of the primers, and enzymatically elongating from the primers to generate an amplification product. Amplification typically requires the presence of deoxyribonucleoside triphosphates, a DNA polymerase enzyme (e.g., Platinum® Taq) and an appropriate buffer and/or co-factors for optimal activity of the polymerase enzyme (e.g., MgCl₂ and/or KCl).

The term “primer” is used herein as known to those skilled in the art and refers to oligomeric compounds, primarily to oligonucleotides but also to modified oligonucleotides that are able to “prime” DNA synthesis by a template-dependent DNA polymerase, i.e., the 3′-end of the, e.g., oligonucleotide provides a free 3′-OH group whereto further “nucleotides” may be attached by a template-dependent DNA polymerase establishing 3′ to 5′ phosphodiester linkage whereby deoxynucleoside triphosphates are used and whereby pyrophosphate is released. Therefore, there is—except possibly for the intended function—no fundamental difference between a “primer”, an “oligonucleotide”, or a “probe”.

The term “hybridizing” refers to the annealing of one or more probes to an amplification product. Hybridization conditions typically include a temperature that is below the melting temperature of the probes but that avoids non-specific hybridization of the probes.

The term “5′ to 3′ nuclease activity” refers to an activity of a nucleic acid polymerase, typically associated with the nucleic acid strand synthesis, whereby nucleotides are removed from the 5′ end of nucleic acid strand.

The term “thermostable polymerase” refers to a polymerase enzyme that is heat stable, i.e., the enzyme catalyzes the formation of primer extension products complementary to a template and does not irreversibly denature when subjected to the elevated temperatures for the time necessary to effect denaturation of double-stranded template nucleic acids. Generally, the synthesis is initiated at the 3′ end of each primer and proceeds in the 5′ to 3′ direction along the template strand. Thermostable polymerases have been isolated from Thermus flavus, T. ruber, T. thermophilus, T. aquaticus, T. lacteus, T. rubens, Bacillus stearothermophilus, and Methanothermus fervidus. Nonetheless, polymerases that are not thermostable also can be employed in PCR assays provided the enzyme is replenished.

The term “complement thereof” refers to nucleic acid that is both the same length as, and exactly complementary to, a given nucleic acid.

The term “extension” or “elongation” when used with respect to nucleic acids refers to when additional nucleotides (or other analogous molecules) are incorporated into the nucleic acids. For example, a nucleic acid is optionally extended by a nucleotide incorporating biocatalyst, such as a polymerase that typically adds nucleotides at the 3′ terminal end of a nucleic acid.

The terms “identical” or percent “identity” in the context of two or more nucleic acid sequences, refer to two or more sequences or subsequences that are the same or have a specified percentage of nucleotides that are the same, when compared and aligned for maximum correspondence, e.g., as measured using one of the sequence comparison algorithms available to persons of skill or by visual inspection. Exemplary algorithms that are suitable for determining percent sequence identity and sequence similarity are the BLAST programs, which are described in, e.g., Altschul et al. (1990) “Basic local alignment search tool” J. Mol. Biol. 215:403-410, Gish et al. (1993) “Identification of protein coding regions by database similarity search” Nature Genet. 3:266-272, Madden et al. (1996) “Applications of network BLAST server” Meth. Enzymol. 266:131-141, Altschul et al. (1997) “Gapped BLAST and PSI-BLAST: a new generation of protein database search programs” Nucleic Acids Res. 25:3389-3402, and Zhang et al. (1997) “PowerBLAST: A new network BLAST application for interactive or automated sequence analysis and annotation” Genome Res. 7:649-656, which are each incorporated herein by reference.

A “modified nucleotide” in the context of an oligonucleotide refers to an alteration in which at least one nucleotide of the oligonucleotide sequence is replaced by a different nucleotide that provides a desired property to the oligonucleotide. Exemplary modified nucleotides that can be substituted in the oligonucleotides described herein include, e.g., a C5-methyl-dC, a C5-ethyl-dC, a C5-methyl-dU, a C5-ethyl-dU, a 2,6-diaminopurine, a C5-propynyl-dC, a C5-propynyl-dU, a C7-propynyl-dA, a C7-propynyl-dG, a C5-propargylamino-dC, a C5-propargylamino-dU, a C7-propargylamino-dA, a C7-propargylamino-dG, a 7-deaza-2-deoxyxanthosine, a pyrazolo-pyrimidine analog, a pseudo-dU, a nitro pyrrole, a nitro indole, 2′-0-methyl Ribo-U, 2′-0-methyl Ribo-C, an N4-ethyl-dC, an N6-methyl-dA, and the like. Many other modified nucleotides that can be substituted in the oligonucleotides are referred to herein or are otherwise known in the art. In certain embodiments, modified nucleotide substitutions modify melting temperatures (Tm) of the oligonucleotides relative to the melting temperatures of corresponding unmodified oligonucleotides. To further illustrate, certain modified nucleotide substitutions can reduce non-specific nucleic acid amplification (e.g., minimize primer dimer formation or the like), increase the yield of an intended target amplicon, and/or the like in some embodiments. Examples of these types of nucleic acid modifications are described in, e.g., U.S. Pat. No. 6,001,611, which is incorporated herein by reference.

A “variant” of a given oligonucleotide may contain one or more nucleotide additions, deletions or substitutions such as one or more nucleotide additions, deletions or substitutions at the 5′ end and/or the 3′ end of the respective sequence of the oligonucleotide. As detailed above, a primer (and/or probe) may be chemically modified, i.e., a primer and/or probe may comprise a modified nucleotide or a non-nucleotide compound. A probe (or a primer) is then a modified oligonucleotide. “Modified nucleotides” (or “nucleotide analogs”) differ from a natural “nucleotide” by some modification but still consist of a base or base-like compound, a pentofuranosyl sugar or a pentofuranosyl sugar-like compound, a phosphate portion or phosphate-like portion, or combinations thereof. For example, a “label” may be attached to the base portion of a “nucleotide” whereby a “modified nucleotide” is obtained. A natural base in a “nucleotide” may also be replaced by, e.g., a 7-desazapurine whereby a “modified nucleotide” is obtained as well. The terms “modified nucleotide” or “nucleotide analog” are used interchangeably in the present application. A “modified nucleoside” (or “nucleoside analog”) differs from a natural nucleoside by some modification in the manner as outlined above for a “modified nucleotide” (or a “nucleotide analog”).

Oligonucleotides including modified oligonucleotides and oligonucleotide analogs that amplify a nucleic acid molecule for example, a nucleic acid molecule encoding the vanA gene or the vanB gene nucleic acid sequences, can be designed using, for example, a computer program such as OLIGO (Molecular Biology Insights Inc., Cascade, Colo.). Important features when designing oligonucleotides to be used as amplification primers include, but are not limited to, an appropriate size amplification product to facilitate detection (e.g., by electrophoresis), similar melting temperatures for the members of a pair of primers, and the length of each primer (i.e., the primers need to be long enough to anneal with sequence-specificity and to initiate synthesis but not so long that fidelity is reduced during oligonucleotide synthesis). Typically, oligonucleotide primers are 8 to 50 nucleotides in length (e.g., 8, 10, 12, 14, 16, 18, 20, 22, 24, 26, 28, 30, 32, 34, 36, 38, 40, 42, 44, 46, 48, or 50 nucleotides in length).

In addition to a set of primers, the methods may use one or more probes in order to detect the presence or absence of the vanA and/or the vanB gene. The term “probe” refers to synthetically or biologically produced nucleic acids (DNA or RNA), which by design or selection, contain specific nucleotide sequences that allow them to hybridize under defined predetermined stringencies specifically (i.e., preferentially) to “target nucleic acids”, in the present case to a vanA (target) nucleic acid and/or to a vanB (target) nucleic acid. A “probe” can be referred to as a “detection probe” meaning that it detects the target nucleic acid.

In some embodiments, the described probes can be labeled with at least one fluorescent label. In one embodiment, the probes can be labeled with a donor fluorescent moiety, e.g., a fluorescent dye, and a corresponding acceptor fluorescent moiety, e.g., a quencher.

Designing oligonucleotides to be used as probes can be performed in a manner similar to the design of primers. Embodiments may use a single probe or a pair of probes for detection of the amplification product. Depending on the embodiment, the probe(s) use may comprise at least one label and/or at least one quencher moiety. As with the primers, the probes usually have similar melting temperatures, and the length of each probe must be sufficient for sequence-specific hybridization to occur but not so long that fidelity is reduced during synthesis. Oligonucleotide probes are generally 15 to 30 (e.g., 16, 18, 20, 21, 22, 23, 24, or 25) nucleotides in length.

Constructs can include vectors each containing one of vanA or vanB primers and probes nucleic acid molecules (e.g., SEQ ID NOs: 1-15). Constructs can be used, for example, as control template nucleic acid molecules. Vectors suitable for use are commercially available and/or produced by recombinant nucleic acid technology methods routine in the art. vanA and vanB nucleic acid molecules can be obtained, for example, by chemical synthesis, direct cloning from vanA and vanB genes, or by PCR amplification.

Constructs suitable for use in the methods typically include, in addition to the vanA and vanB nucleic acid molecules (e.g., a nucleic acid molecule that contains one or more sequences of SEQ ID NOs: 1-15), sequences encoding a selectable marker (e.g., an antibiotic resistance gene) for selecting desired constructs and/or transformants, and an origin of replication. The choice of vector systems usually depends upon several factors, including, but not limited to, the choice of host cells, replication efficiency, selectability, inducibility, and the ease of recovery.

Constructs containing vanA and vanB nucleic acid molecules can be propagated in a host cell. As used herein, the term host cell is meant to include prokaryotes and eukaryotes such as yeast, plant and animal cells. Prokaryotic hosts may include E. coli, Salmonella typhimurium, Serratia marcescens, and Bacillus subtilis. Eukaryotic hosts include yeasts such as S. cerevisiae, S. pombe, Pichia pastoris, mammalian cells such as COS cells or Chinese hamster ovary (CHO) cells, insect cells, and plant cells such as Arabidopsis thaliana and Nicotiana tabacum. A construct can be introduced into a host cell using any of the techniques commonly known to those of ordinary skill in the art. For example, calcium phosphate precipitation, electroporation, heat shock, lipofection, microinjection, and viral-mediated nucleic acid transfer are common methods for introducing nucleic acids into host cells. In addition, naked DNA can be delivered directly to cells (see, e.g., U.S. Pat. Nos. 5,580,859 and 5,589,466).

### Polymerase Chain Reaction (PCR)

U.S. Pat. Nos. 4,683,202, 4,683,195, 4,800,159, and 4,965,188 disclose conventional PCR techniques. PCR typically employs two oligonucleotide primers that bind to a selected nucleic acid template (e.g., DNA or RNA). Primers useful in some embodiments include oligonucleotides capable of acting as points of initiation of nucleic acid synthesis within the described mecA/mecC-MRSA nucleic acid sequences (e.g., SEQ ID NOs: 1, 2, 4, 5, and 6). A primer can be purified from a restriction digest by conventional methods, or it can be produced synthetically. The primer is preferably single-stranded for maximum efficiency in amplification, but the primer can be double-stranded. Double-stranded primers are first denatured, i.e., treated to separate the strands. One method of denaturing double stranded nucleic acids is by heating.

If the template nucleic acid is double-stranded, it is necessary to separate the two strands before it can be used as a template in PCR. Strand separation can be accomplished by any suitable denaturing method including physical, chemical or enzymatic means. One method of separating the nucleic acid strands involves heating the nucleic acid until it is predominately denatured (e.g., greater than 50%, 60%, 70%, 80%, 90% or 95% denatured). The heating conditions necessary for denaturing template nucleic acid will depend, e.g., on the buffer salt concentration and the length and nucleotide composition of the nucleic acids being denatured, but typically range from about 90° C. to about 105° C. for a time depending on features of the reaction such as temperature and the nucleic acid length. Denaturation is typically performed for about 30 sec to 4 min (e.g., 1 min to 2 min 30 sec, or 1.5 min).

If the double-stranded template nucleic acid is denatured by heat, the reaction mixture is allowed to cool to a temperature that promotes annealing of each primer to its target sequence on the described nucleic acid molecules. The temperature for annealing is usually from about 35° C. to about 65° C. (e.g., about 40° C. to about 60° C.; about 45° C. to about 50° C.). Annealing times can be from about 10 sec to about 1 min (e.g., about 20 sec to about 50 sec; about 30 sec to about 40 sec). The reaction mixture is then adjusted to a temperature at which the activity of the polymerase is promoted or optimized, i.e., a temperature sufficient for extension to occur from the annealed primer to generate products complementary to the template nucleic acid. The temperature should be sufficient to synthesize an extension product from each primer that is annealed to a nucleic acid template, but should not be so high as to denature an extension product from its complementary template (e.g., the temperature for extension generally ranges from about 40° C. to about 80° C. (e.g., about 50° C. to about 70° C.; about 60° C.). Extension times can be from about 10 sec to about 5 min (e.g., about 30 sec to about 4 min; about 1 min to about 3 min; about 1 min 30 sec to about 2 min). PCR assays can employ vanA gene and vanB gene nucleic acid such as RNA or DNA (cDNA). The template nucleic acid need not be purified; it may be a minor fraction of a complex mixture, such as vanA and/or vanB nucleic acid contained in biological samples. VanA vanB nucleic acid molecules may be extracted from a biological sample by routine techniques such as those described in Diagnostic Molecular Microbiology: Principles and Applications (Persing et al. (eds), 1993, American Society for Microbiology, Washington D.C.). Nucleic acids can be obtained from any number of sources, such as plasmids, or natural sources including bacteria, yeast, viruses, organelles, or higher organisms such as plants or animals.

The oligonucleotide primers are combined with PCR reagents under reaction conditions that induce primer extension. For example, chain extension reactions generally include 50 mM KCl, 10 mM Tris-HCl (pH 8.3), 15 mM MgCl₂, 0.001% (w/v) gelatin, 0.5-1.0 μg denatured template DNA, 50 pmoles of each oligonucleotide primer, 2.5 U of Taq polymerase, and 10% DMSO). The reactions usually contain 150 to 320 μM each of dATP, dCTP, dTTP, dGTP, or one or more analogs thereof.

The newly synthesized strands form a double-stranded molecule that can be used in the succeeding steps of the reaction. The steps of strand separation, annealing, and elongation can be repeated as often as needed to produce the desired quantity of amplification products corresponding to the target nucleic acid molecules. The limiting factors in the reaction are the amounts of primers, thermostable enzyme, and nucleoside triphosphates present in the reaction. The cycling steps (i.e., denaturation, annealing, and extension) are preferably repeated at least once. For use in detection, the number of cycling steps will depend, e.g., on the nature of the sample. If the sample is a complex mixture of nucleic acids, more cycling steps will be required to amplify the target sequence sufficient for detection. Generally, the cycling steps are repeated at least about 20 times, but may be repeated as many as 40, 60, or even 100 times.

### Fluorescence Resonance Energy Transfer (FRET)

FRET technology (see, for example, U.S. Pat. Nos. 4,996,143, 5,565,322, 5,849,489, and 6,162,603) is based on a concept that when a donor fluorescent moiety and a corresponding acceptor fluorescent moiety are positioned within a certain distance of each other, energy transfer takes place between the two fluorescent moieties that can be visualized or otherwise detected and/or quantitated. The donor typically transfers the energy to the acceptor when the donor is excited by light radiation with a suitable wavelength. The acceptor typically re-emits the transferred energy in the form of light radiation with a different wavelength. In certain systems, non-fluorescent energy can be transferred between donor and acceptor moieties, by way of biomolecules that include substantially non-fluorescent donor moieties (see, for example, U.S. Pat. No. 7,741,467).

In one example, a oligonucleotide probe can contain a donor fluorescent moiety and a corresponding quencher, which may or not be fluorescent, and which dissipates the transferred energy in a form other than light. When the probe is intact, energy transfer typically occurs between the two fluorescent moieties such that fluorescent emission from the donor fluorescent moiety is quenched. During an extension step of a polymerase chain reaction, a probe bound to an amplification product is cleaved by the 5′ to 3′ nuclease activity of, e.g., a Taq Polymerase such that the fluorescent emission of the donor fluorescent moiety is no longer quenched. Exemplary probes for this purpose are described in, e.g., U.S. Pat. Nos. 5,210,015, 5,994,056, and 6,171,785. Commonly used donor-acceptor pairs include the FAM-TAMRA pair. Commonly used quenchers are DABCYL and TAMRA. Commonly used dark quenchers include BlackHole Quenchers™ (BHQ), (Biosearch Technologies, Inc., Novato, Cal.), Iowa Black™, (Integrated DNA Tech., Inc., Coralville, Iowa), BlackBerry™ Quencher 650 (BBQ-650), (Berry & Assoc., Dexter, Mich.).

In another example, two oligonucleotide probes, each containing a fluorescent moiety, can hybridize to an amplification product at particular positions determined by the complementarity of the oligonucleotide probes to the target nucleic acid sequence. Upon hybridization of the oligonucleotide probes to the amplification product nucleic acid at the appropriate positions, a FRET signal is generated. Hybridization temperatures can range from about 35° C. to about 65° C. for about 10 sec to about 1 min.

Fluorescent analysis can be carried out using, for example, a photon counting epifluorescent microscope system (containing the appropriate dichroic mirror and filters for monitoring fluorescent emission at the particular range), a photon counting photomultiplier system, or a fluorimeter. Excitation to initiate energy transfer, or to allow direct detection of a fluorophore, can be carried out with an argon ion laser, a high intensity mercury (Hg) arc lamp, a fiber optic light source, or other high intensity light source appropriately filtered for excitation in the desired range. As used herein with respect to donor and corresponding acceptor fluorescent moieties “corresponding” refers to an acceptor fluorescent moiety having an absorbance spectrum that overlaps the emission spectrum of the donor fluorescent moiety. The wavelength maximum of the emission spectrum of the acceptor fluorescent moiety should be at least 100 nm greater than the wavelength maximum of the excitation spectrum of the donor fluorescent moiety. Accordingly, efficient non-radiative energy transfer can be produced there between.

Fluorescent donor and corresponding acceptor moieties are generally chosen for (a) high efficiency Forster energy transfer; (b) a large final Stokes shift (>100 nm); (c) shift of the emission as far as possible into the red portion of the visible spectrum (>600 nm); and (d) shift of the emission to a higher wavelength than the Raman water fluorescent emission produced by excitation at the donor excitation wavelength. For example, a donor fluorescent moiety can be chosen that has its excitation maximum near a laser line (for example, Helium-Cadmium 442 nm or Argon 488 nm), a high extinction coefficient, a high quantum yield, and a good overlap of its fluorescent emission with the excitation spectrum of the corresponding acceptor fluorescent moiety. A corresponding acceptor fluorescent moiety can be chosen that has a high extinction coefficient, a high quantum yield, a good overlap of its excitation with the emission of the donor fluorescent moiety, and emission in the red part of the visible spectrum (>600 nm).

Representative donor fluorescent moieties that can be used with various acceptor fluorescent moieties in FRET technology include fluorescein, Lucifer Yellow, B-phycoerythrin, 9-acridineisothiocyanate, Lucifer Yellow VS, 4-acetamido-4′-isothio-cyanatostilbene-2,2′-disulfonic acid, 7-diethylamino-3-(4′-isothiocyanatophenyl)-4-methylcoumarin, succinimdyl 1-pyrenebutyrate, and 4-acetamido-4′-isothiocyanatostilbene-2,2′-disulfonic acid derivatives. Representative acceptor fluorescent moieties, depending upon the donor fluorescent moiety used, include LC Red 640, LC Red 705, Cy5, Cy5.5, Lissamine rhodamine B sulfonyl chloride, tetramethyl rhodamine isothiocyanate, rhodamine x isothiocyanate, erythrosine isothiocyanate, fluorescein, diethylenetriamine pentaacetate, or other chelates of Lanthanide ions (e.g., Europium, or Terbium). Donor and acceptor fluorescent moieties can be obtained, for example, from Molecular Probes (Junction City, Oreg.) or Sigma Chemical Co. (St. Louis, Mo.).

The donor and acceptor fluorescent moieties can be attached to the appropriate probe oligonucleotide via a linker arm. The length of each linker arm is important, as the linker arms will affect the distance between the donor and acceptor fluorescent moieties. The length of a linker arm can be the distance in Angstroms (Å) from the nucleotide base to the fluorescent moiety. In general, a linker arm is from about 10 Å to about 25 Å. The linker arm may be of the kind described in WO 84/03285. WO 84/03285 also discloses methods for attaching linker arms to a particular nucleotide base, and also for attaching fluorescent moieties to a linker arm.

An acceptor fluorescent moiety, such as an LC Red 640, can be combined with an oligonucleotide which contains an amino linker (e.g., C6-amino phosphoramidites available from ABI (Foster City, Calif.) or Glen Research (Sterling, VA)) to produce, for example, LC Red 640-labeled oligonucleotide. Frequently used linkers to couple a donor fluorescent moiety such as fluorescein to an oligonucleotide include thiourea linkers (FITC-derived, for example, fluorescein-CPG's from Glen Research or ChemGene (Ashland, Mass.)), amide-linkers (fluorescein-NHS-ester-derived, such as CX-fluorescein-CPG from BioGenex (San Ramon, Calif.)), or 3′-amino-CPGs that require coupling of a fluorescein-NHS-ester after oligonucleotide synthesis.

### Detection of vanA and vanB Resistance Genes

The present disclosure provides methods for detecting the presence or absence of vanA and/or vanB gene in a biological or non-biological sample. Methods provided avoid problems of sample contamination, false negatives, and false positives. The methods include performing at least one cycling step that includes amplifying a portion of vanA and/or vanB target nucleic acid molecules from a sample using a plurality of pairs of vanA and/or vanB primers, and a FRET detecting step. Multiple cycling steps are performed, preferably in a thermocycler. Methods can be performed using the vanA and vanB primers and probes to detect the presence of vanA gene and/or vanB gene, and the detection of vanA and or vanB in the assay indicates the presence of vanA and/or vanB in the sample.

As described herein, amplification products can be detected using labeled hybridization probes that take advantage of FRET technology. One FRET format utilizes TaqMan® technology to detect the presence or absence of an amplification product, and hence, the presence or absence of mecA/mecC-MRSA. TaqMan® technology utilizes one single-stranded hybridization probe labeled with, e.g., one fluorescent dye and one quencher, which may or may not be fluorescent. When a first fluorescent moiety is excited with light of a suitable wavelength, the absorbed energy is transferred to a second fluorescent moiety according to the principles of FRET. The second fluorescent moiety is generally a quencher molecule. During the annealing step of the PCR reaction, the labeled hybridization probe binds to the target DNA (i.e., the amplification product) and is degraded by the 5′ to 3′ nuclease activity of, e.g., the Taq Polymerase during the subsequent elongation phase. As a result, the fluorescent moiety and the quencher moiety become spatially separated from one another. As a consequence, upon excitation of the first fluorescent moiety in the absence of the quencher, the fluorescence emission from the first fluorescent moiety can be detected. By way of example, an ABI PRISM® 7700 Sequence Detection System (Applied Biosystems) uses TaqMan® technology, and is suitable for performing the methods described herein for detecting the presence or absence of vanA and/or vanB in the sample.

Molecular beacons in conjunction with FRET can also be used to detect the presence of an amplification product using the real-time PCR methods. Molecular beacon technology uses a hybridization probe labeled with a first fluorescent moiety and a second fluorescent moiety. The second fluorescent moiety is generally a quencher, and the fluorescent labels are typically located at each end of the probe. Molecular beacon technology uses a probe oligonucleotide having sequences that permit secondary structure formation (e.g., a hairpin). As a result of secondary structure formation within the probe, both fluorescent moieties are in spatial proximity when the probe is in solution. After hybridization to the target nucleic acids (i.e., amplification products), the secondary structure of the probe is disrupted and the fluorescent moieties become separated from one another such that after excitation with light of a suitable wavelength, the emission of the first fluorescent moiety can be detected.

Another common format of FRET technology utilizes two hybridization probes. Each probe can be labeled with a different fluorescent moiety and are generally designed to hybridize in close proximity to each other in a target DNA molecule (e.g., an amplification product). A donor fluorescent moiety, for example, fluorescein, is excited at 470 nm by the light source of the LightCycler® Instrument. During FRET, the fluorescein transfers its energy to an acceptor fluorescent moiety such as LightCycler®-Red 640 (LC Red 640) or LightCycler®-Red 705 (LC Red 705). The acceptor fluorescent moiety then emits light of a longer wavelength, which is detected by the optical detection system of the LightCycler® instrument. Efficient FRET can only take place when the fluorescent moieties are in direct local proximity and when the emission spectrum of the donor fluorescent moiety overlaps with the absorption spectrum of the acceptor fluorescent moiety. The intensity of the emitted signal can be correlated with the number of original target DNA molecules. If amplification of target nucleic acid occurs and an amplification product is produced, the step of hybridizing results in a detectable signal based upon FRET between the members of the pair of probes.

Generally, the presence of FRET indicates the presence of the target sequence in the sample, and the absence of FRET indicates the absence of the target sequence in the sample. Inadequate specimen collection, transportation delays, inappropriate transportation conditions, or use of certain collection swabs (calcium alginate or aluminum shaft) are all conditions that can affect the success and/or accuracy of a test result, however. Using the methods disclosed herein, detection of FRET within, e.g., 45 cycling steps is indicative of a vanA and/or vanB infection.

Representative biological samples that can be used in practicing the methods include, but are not limited to dermal swabs, nasal swabs, wound swabs, blood cultures, skin, and soft tissue infections. Collection and storage methods of biological samples are known to those of skill in the art. Biological samples can be processed (e.g., by nucleic acid extraction methods and/or kits known in the art) to release target gene nucleic acid or in some cases, the biological sample can be contacted directly with the PCR reaction components and the appropriate oligonucleotides.

Melting curve analysis is an additional step that can be included in a cycling profile. Melting curve analysis is based on the fact that DNA melts at a characteristic temperature called the melting temperature (Tm), which is defined as the temperature at which half of the DNA duplexes have separated into single strands. The melting temperature of a DNA depends primarily upon its nucleotide composition. Thus, DNA molecules rich in G and C nucleotides have a higher Tm than those having an abundance of A and T nucleotides. By detecting the temperature at which signal is lost, the melting temperature of probes can be determined. Similarly, by detecting the temperature at which signal is generated, the annealing temperature of probes can be determined. The melting temperature(s) of the probes from the amplification products can confirm the presence or absence of the target sequence in the sample.

Within each thermocycler run, control samples can be cycled as well. Positive control samples can amplify target nucleic acid control template (other than described amplification products of target genes) using, for example, control primers and control probes. Positive control samples can also amplify, for example, a plasmid construct containing the target nucleic acid molecules. Such a plasmid control can be amplified internally (e.g., within the sample) or in a separate sample run side-by-side with the patients' samples using the same primers and probe as used for detection of the intended target. Such controls are indicators of the success or failure of the amplification, hybridization, and/or FRET reaction. Each thermocycler run can also include a negative control that, for example, lacks target template DNA. Negative control can measure contamination. This ensures that the system and reagents would not give rise to a false positive signal. Therefore, control reactions can readily determine, for example, the ability of primers to anneal with sequence-specificity and to initiate elongation, as well as the ability of probes to hybridize with sequence-specificity and for FRET to occur.

In an embodiment, the methods include steps to avoid contamination. For example, an enzymatic method utilizing uracil-DNA glycosylase is described in U.S. Pat. Nos. 5,035,996, 5,683,896 and 5,945,313 to reduce or eliminate contamination between one thermocycler run and the next.

Conventional PCR methods in conjunction with FRET technology can be used to practice the methods. In one embodiment, a LightCycler® instrument is used. The following patent applications describe real-time PCR as used in the LightCycler® technology: WO 97/46707, WO 97/46714, and WO 97/46712.

The LightCycler® can be operated using a PC workstation and can utilize a Windows NT operating system. Signals from the samples are obtained as the machine positions the capillaries sequentially over the optical unit. The software can display the fluorescence signals in real-time immediately after each measurement. Fluorescent acquisition time is 10-100 milliseconds (msec). After each cycling step, a quantitative display of fluorescence vs. cycle number can be continually updated for all samples. The data generated can be stored for further analysis.

As an alternative to FRET, an amplification product can be detected using a double-stranded DNA binding dye such as a fluorescent DNA binding dye (e.g., SYBR® Green or SYBR® Gold (Molecular Probes)). Upon interaction with the double-stranded nucleic acid, such fluorescent DNA binding dyes emit a fluorescence signal after excitation with light at a suitable wavelength. A double-stranded DNA binding dye such as a nucleic acid intercalating dye also can be used. When double-stranded DNA binding dyes are used, a melting curve analysis is usually performed for confirmation of the presence of the amplification product.

It is understood that the embodiments of the present disclosure are not limited by the configuration of one or more commercially available instruments.

### Articles of Manufacture/Kits

Embodiments of the present disclosure further provide for articles of manufacture or kits to detect the vanA gene and the van B gene. An article of manufacture can include primers and probes used to detect vanA and/or vanB, together with suitable packaging materials. Representative primers and probes for detection of vanA and/or vanB are capable of hybridizing to vanA and/or vanB target nucleic acid molecules. In addition, the kits may also include suitably packaged reagents and materials needed for DNA immobilization, hybridization, and detection, such solid supports, buffers, enzymes, and DNA standards. Methods of designing primers and probes are disclosed herein, and representative examples of primers and probes that amplify and hybridize to mecAlmecC-MRSA target nucleic acid molecules are provided.

Articles of manufacture can also include one or more fluorescent moieties for labeling the probes or, alternatively, the probes supplied with the kit can be labeled. For example, an article of manufacture may include a donor and/or an acceptor fluorescent moiety for labeling the vanA and vanB probes. Examples of suitable FRET donor fluorescent moieties and corresponding acceptor fluorescent moieties are provided above.

Articles of manufacture can also contain a package insert or package label having instructions thereon for using the vanA and/or vanB primers and probes to detect vanA and/or vanB in a sample. Articles of manufacture may additionally include reagents for carrying out the methods disclosed herein (e.g., buffers, polymerase enzymes, co-factors, or agents to prevent contamination). Such reagents may be specific for one of the commercially available instruments described herein.

Embodiments of the present disclosure will be further described in the following examples, which do not limit the scope of the invention described in the claims.

### EXAMPLES

The following examples and figures are provided to aid the understanding of the subject matter, the true scope of which is set forth in the appended claims. It is understood that modifications can be made in the procedures set forth without departing from the spirit of the invention.

### Example I

TABLE I
vanA/vanB Primers and Probes

| SEQ | | | | |
|---|---|---|---|---|
| ID NO | NAME | DESCRIPTION | SEQUENCE | MODIFICATION |
| 1 | PLN_JS_vanA_F2 | vanA forward | GGAATTACGAAATCTGGTGT | |
| | | primer | ATGGAAA | |
| 2 | SEGP3128_TBB | vanA forward | GGAATTACGAAATCTGGTGT | J = t-butylbenzyl-dA |
| | | primer | ATGGAAJ | |
| 3 | PLN JS_vanA_R2 | vanA reverse | GGTTCTTTTTAACAAGTAAT | |
| | | primer | CCGTGCA | |
| 4 | SEGP3129_TBB | vanA reverse | GGTTCTTTTTAACAAGTAAT | J = t-butylbenzyl-dA |
| | | primer | CCGTGCJ | |
| 5 | SEGP3130_vanA_ATTO | vanA probe | <ATTO>TGGGAAAACQGACA | <ATTO> = ATTO425 |
| | | | ATTGCTATTCAGCTGTACTCT | N dye, Q = BHQ2, |
| | | | CGp | p = 3′phosphate |
| 6 | PLN_JS_Enterococcus_ | vanB forward | GCAGGTGTGGGATTGCTTTA | |
| | vanB_F2 | primer | TCAA | |
| 7 | SEGP3124_TBB | vanB forward | GCAGGTGTGGGATTGCTTTA | J = t-butylbenzyl-dA |
| | | primer | TCAJ | |
| 8 | PLN_JS_Enterococcus_ | vanB reverse | CATTTTATTCGCACTGTCCCG | |
| | vanB_R2 | primer | CTAA | |
| 9 | SEGP3135_TBB | vanB reverse | CATTTTATTCGCACTGTCCCG | J = t-butylbenzyl-dA |
| | | primer | CTAJ | |
| 10 | SEGP3136_vanB_ATTO | vanB probe | <ATTO>TGATTGCGAQTAAT | <ATTO> = ATTO425 |
| | | | GGTTGTATTCAGCCTTTTATG | N dye, Q = BHQ2, |
| | | | CGCp | p = 3′phosphate |
| 11 | PLS_JS_Enterococcus_ | vanB forward | GCACAGCAATTTGTGTCTTA | |
| | vanB_F3 | primer | TTTCAGA | |
| 12 | SEGP3137_TBB | vanB forward | GCACAGCAATTTGTGTCTTA | J = t-butylbenzyl-dA |
| | | primer | TTTCAGJ | |
| 13 | PLS_JS_Enterococcus_ | vanB reverse | CTCAAAGGATTGGTTATTGT | |
| | vanB_R3 | primer | AGTGAAACA | |
| 14 | SEGP3138_TBB | vanB reverse | CTCAAAGGATTGGTTATTGT | J = t-butylbenzyl-dA |
| | | primer | AGTGAAACJ | |
| 15 | SEGP3139_vanB_ATTO | vanB probe | <ATTO>TCAGCCATTQGGTG | <ATTO> = ATTO425 |
| | | | GAACTGATTCAGAATAGCGA | N dye, Q = BHQ2, |
| | | | TAGp | p = 3`phosphate |

### VanA and vanB Primer and Probe Sequences

TABLE I shows the primers and probes that are used in the PCR assays for the detection of the vanA gene and the vanB gene.

### Example 2

### PCR Experimental Conditions

Real-time PCR detection of the vanA target gene or the vanB target gene were performed using either the cobas® 4800 system or the cobas® 6800/8800 systems platforms (Roche Molecular Systems, Inc., Pleasanton, CA). The final concentrations of the amplification reagents are shown below:

TABLE II
PCR Amplification Reagents

| Master Mix Component | Final Conc (50 uL) | |
|---|---|---|
| DMSO | 0-5.4 | % |
| NaN3 | 0.027-0.030 | % |
| Potassium acetate | 120.0 | mM |
| Glycerol | 3.0 | % |
| Tween 20 | 0.02 | % |
| EDTA | 0-43.9 | uM |
| Tricine | 60.0 | mM |
| Aptamer | 0.18-0.22 | uM |
| UNG Enzyme | 5.0-10.0 | U |
| Z05-SP-PZ Polymerase | 30.0-45.0 | U |
| dATP | 400.0-521.70 | uM |
| dCTP | 400.0-521.70 | uM |
| dGTP | 400.0-521.70 | uM |
| dUTP | 800.0-1043.40 | uM |
| Forward primer oligonucleotides | 0.15-0.50 | μM |
| Reverse primer oligonucleotides | 0.15-0.50 | μM |
| Probe oligonucleotides | 0.10 | μM |
| Manganese Acetate | 3.30-3.80 | mM |

The following table shows the typical thermoprofile used for PCR amplification reaction:

TABLE III
PCR Thermoprofile

| | Target | Acquisition | Hold | Ramp Rate | | Analysis |
|---|---|---|---|---|---|---|
| Program Name | (° C.) | Mode | (hh:mm:ss) | (° C./s) | Cycles | Mode |
| Pre-PCR | 50 | None | 00:02:00 | 4.4 | 1 | None |
| | 94 | None | 00:00:05 | 4.4 | | |
| | 55 | None | 00:02:00 | 2.2 | | |
| | 60 | None | 00:06:00 | 4.4 | | |
| | 65 | None | 00:04:00 | 4.4 | | |
| 1^{st} Measurement | 95 | None | 00:00:05 | 4.4 | 5 | Quantification |
| | 55 | Single | 00:00:30 | 2.2 | | |
| 2^{nd} Measurement | 91 | None | 00:00:05 | 4.4 | 45 | Quantification |
| | 58 | Single | 00:00:25 | 2.2 | | |
| Cooling | 40 | None | 00:02:00 | 2.2 | 1 | None |

The Pre-PCR program comprised initial denaturing and incubation at 55° C., 60° C. and 65° C. for reverse transcription of RNA templates. Incubating at three temperatures combines the advantageous effects that at lower temperatures slightly mismatched target sequences (such as genetic variants of an organism) are also transcribed, while at higher temperatures the formation of RNA secondary structures is suppressed, thus leading to a more efficient transcription. PCR cycling was divided into two measurements, wherein both measurements apply a one-step setup (combining annealing and extension). The first 5 cycles at 55° C. allow for an increased inclusivity by pre-amplifying slightly mismatched target sequences, whereas the 45 cycles of the second measurement provide for an increased specificity by using an annealing/extension temperature of 58° C.

### Example 3

### Performance of vanA and vanB Primers and Probes in PCR Assay

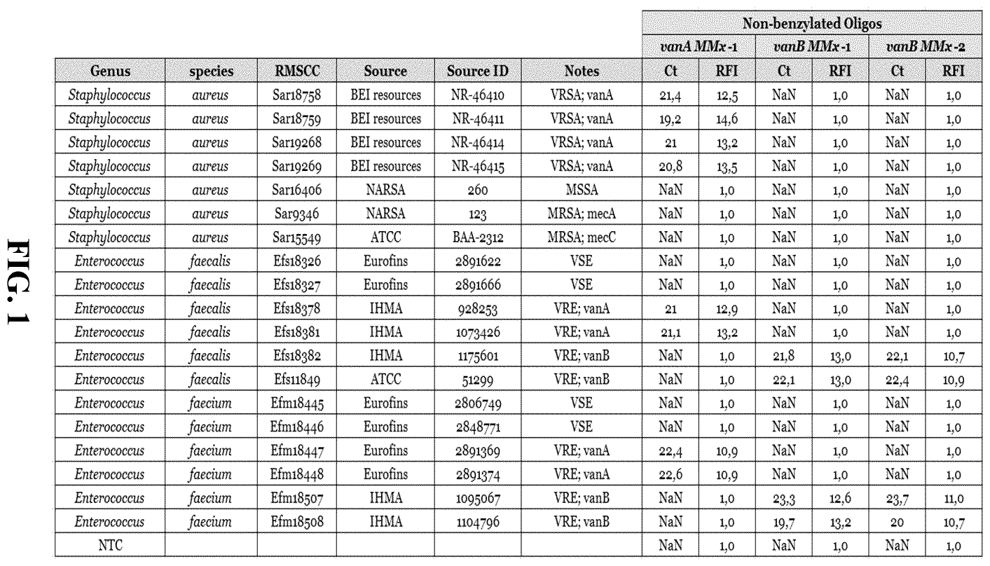

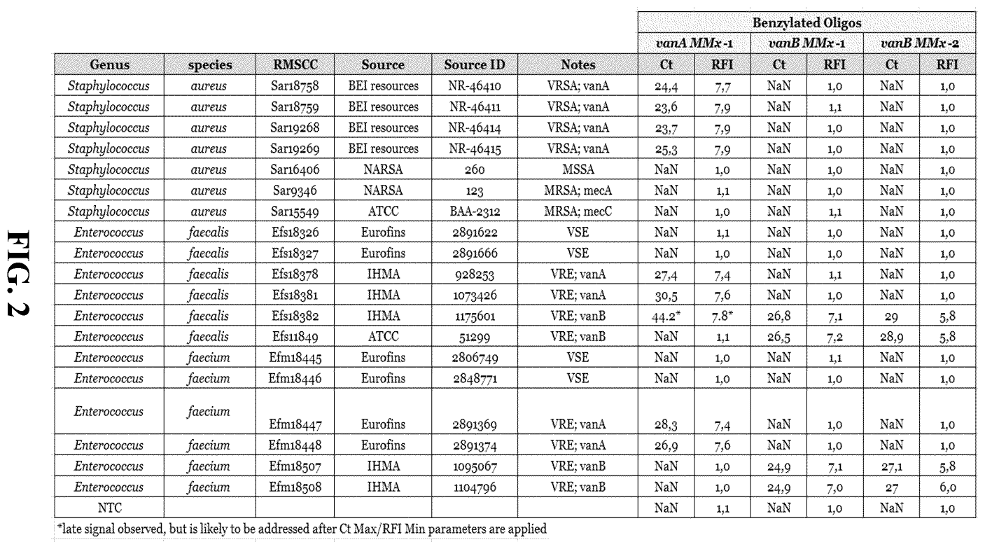

PCR experiments using either non-benzylated or benzylated primers for the amplification of the vanA gene and the vanB gene in various strains of Staphylococcus aureus, Enterococcus faecalis, and Enterococcus faecium, followed by detection using the vanA or vanB gene probes. FIG. 1 shows the results using non-benzylated primers which are SEQ ID NOs: 1 and 3 in the vanA MMx-1 column with detection by the vanA probe of SEQ ID NO: 5; SEQ ID NOs: 6 and 8 in the vanB MMx-1 column with detection by the vanB probe of SEQ ID NO: 10; and SEQ ID NOs: 11 and 13 in the vanB MMx-2 column with detection by the vanB probe of SEQ ID NO: 15. The presence and detection of the respective amplification products are shown as Ct values. FIG. 2 shows the results using benzylated primers which are SEQ ID NOs: 2 and 4 in the vanA MMx-1 column with detection by the vanA probe of SEQ ID NO: 5; SEQ ID NOs: 7 and 9 in the vanB MMx-1 column with detection by the vanB probe of SEQ ID NO: 10; and SEQ ID NOs: 12 and 14 in the vanB

MMx-2 column with detection by the vanB probe of SEQ ID NO: 15. The presence and detection of the respective amplification products are shown as Ct values.

No significant differences were observed in the performance between the benzylated and the non-benzylated primers. More importantly, the one set of vanA primers and probe and two sets of vanB primers and probes were all able to amplify and detect their respective target nucleic acids in a specific manner.

### Example 4

### Multiplex PCR Assay For Detection of VRSA and MRSA

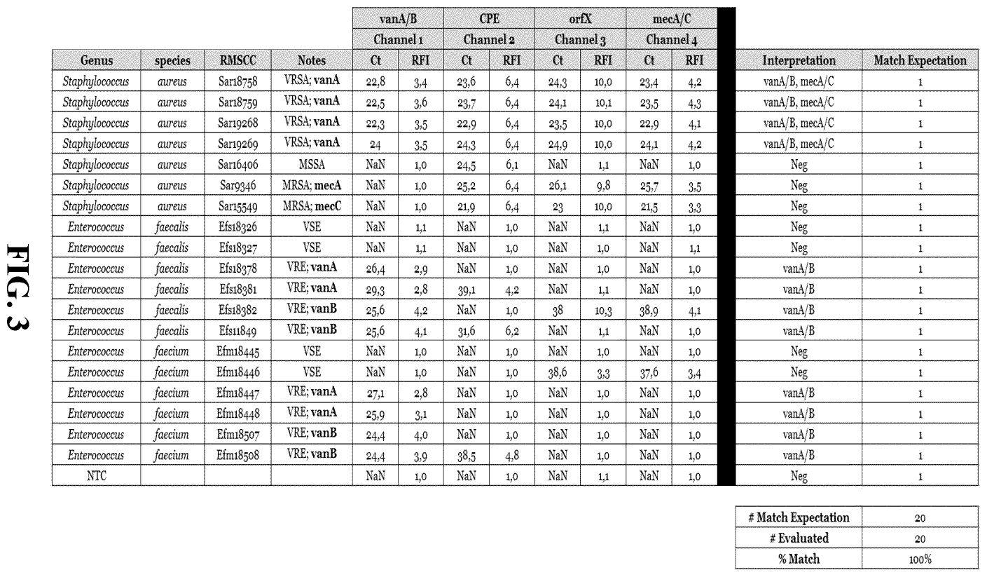

A multiplex PCR assay for detection of both Vancomycin-Resistant Staphylococcus aureus (VRSA) by targeting vanA and vanB genes and Methicillin-Resistant Staphylococcus aureus (MRSA) by targeting the mecA and mecC genes as well as the SCCmec OrfX right extremity junction (OrfX) was set up and the primers and probes in this assay are shown in TABLE IV. The results of the PCR assay using these sets of primers and probes against various Staphylococcus aureus and Enterococcus strains are shown on FIG. 3

TABLE IV
Primers and Probes for Detecting VRSA and MRSA

| SEQ | | | | |
|---|---|---|---|---|
| ID NO | NAME | DESCRIPTION | SEQUENCE | MODIFICATION |
| 2 | SEGP3128_TBB | vanA forward | GGAATTACGAAATCTGGTGTAT | J = t-butylbenzyl-d.A |
| | | primer | GGAAJ | |
| 4 | SEGP3129_TBB | vanA reverse | GGTTCTTTTTAACAAGTAATCCG | J = t-butylbenzyl-d.A |
| | | primer | TGCJ | |
| 5 | SEGP3130_vanA_ | vanA probe | <ATTO>TGGGAAAACQGACAAT | <ATTO> = ATTO42 |
| | ATTO | | TGCTATTCAGCTGTACTCTCGp | 5N dye, Q = BHQ2, |
| | | | | p = 3′phosphate |
| 7 | SEGP3134_TBB | vanB forward | GCAGGTGTGGGATTGCTTTATC | J = t-butylbenzyl-d.A |
| | | primer | AJ | |
| 9 | SEGP3135_TBB | vanB reverse | CATTTTATTCGCACTGTCCCGCT | J = t-butylbenzyl-dA |
| | | primer | AJ | |
| 10 | SEGP3136_vanB_ | vanB probe | <ATTO>TGATTGCGAQTAATGGT | <ATTO> = ATTO42 |
| | ATTO | | TGTATTCAGCCTTTTATGCGCp | 5N dye, Q = BHQ2, |
| | | | | p = 3′phosphate |
| 16 | JJ152BBC | CPE forward | AAGATAAGCTTATTGAACAAGG | K = t-butylbenzyl-dC |
| | | primer | ACATK | |
| 17 | JJ157BBC | CPE reverse primer | CTTGAGGTGAATTGTIGTGAAC | K = t-butylbenzyl-dC |
| | | | K | |
| 18 | SAPS | CPE probe | FTTAGGAATZCAATTATGGAAGT | F-5′fluorescein, |
| | | | CGACCTCGT3IABKFQ | Z = ZEN, 3IABKFQ = |
| | | | | quencher |
| 19 | ORFX_F1 | orfX forward | CAGCAAAATGACATTCCCACAT | J = t-butylbenzyl-d.A |
| | | primer | CJ | |
| 20 | ORFX_F2 | orfX forward | CAGCAAAATGACATTTCCACAC | J = t-butylbenzyl-d.A |
| | | primer | CJ | |
| 21 | ORFX_R-2 | orfX reverse | CCTCCACATCTCATTAAATTTTT | J = t-butylbenzyl-dA |
| | | primer | AAATTATACACAJ | |
| 22 | ORFX_R-4 | orfX reverse | TTCTTCAAAGATTTGAGCTAATT | J = t-butylbenzyl-dA |
| | | primer | TAATAATTTTCTCJ | |
| 23 | ORFX_R-5 | orfX reverse | GGAGGCTAACTATGTCAAAAAT | J = t-butylbenzyl-dA |
| | | primer | CATGAJ | |
| 24 | ORFX_R-1-2N1 | orfX reverse | GAGGTTAAAAAAACCGCATCAT | J = t-butylbenzyl-dA |
| | | primer | TTGTGJ | |
| 25 | ORFX_A_SIMA | orfX probe | HGATGCGGZGTTGTGTTAATTG | H = HEX dye, |
| | | | AACAAGTGTATAGAGCATT3IAB | Z = ZEN, |
| | | | kFQ | 3IABKFQ = quenche |
| | | | | r |
| 26 | MECA_F1006 | mecA forward | ATGATGCAGTTATTGGTAAAAA | K = t-butylbenzyl-dC |
| | | primer | GGGACTK | |
| 27 | MECA_R1077 | mecA reverse | TGTATGTGCGATTGTATTGCTAT | K = t-butylbenzyl-dC |
| | | primer | TATCGTK | |
| 28 | MECA_P2_TAO9 | mecA probe | <LCR_640>CTCCAACAT<TAO>G | <LCR_640> : = |
| | | | AAGATGGCTATCGTGTCACAAT | Fluorophore, |
| | | | CGT<IB_RQ> | <TAO> = Quencher |
| | | | | <IB_RQ> = 3′ |
| | | | | Blocker |
| 29 | MECC_F1_TBB | mecC forward | ACGGCAATATCGATTTAAAGCA | J = t-butylbenzyl-dA |
| | | primer | AGCAATJ | |
| 30 | MECC_R1_TBB | mecC reverse | TGCTTTATAAAAGGGATAATCA | J = t-butylbenzyl-dA |
| | | primer | CTCGGGATJ | |
| 31 | MECCP1_LCR91 | mecC probe | <LCR_640>TGAGCAAGGT<TAO> | <LCR_640> = |
| | | | ATGCAAGATTTGGGAATCGGTG | Fluorophore, <TAO> = |
| | | | A<IB_RQ> | Quencher <IB_RQ> = 3′ |
| | | | | Blocker |

1. A method for detecting bacteria having a vanA gene and/or a vanB gene in a sample, the method comprising:
performing an amplifying step comprising contacting the sample with a set of vanA forward and reverse primers and a set of vanB forward and reverse primers to produce an amplification product if the vanA gene and/or the vanB gene is present in the sample;
performing a hybridizing step including contacting the amplification product with one or more detectable vanA probes and one or more detectable vanB probes; and
detecting the presence or absence of the amplification product, wherein the presence of the amplified product is indicative of the presence of the vanA and/or vanB gene in the sample and wherein the absence of the amplified product is indicative of the absence of the vanA gene and/or vanB gene in the sample;
wherein the set of vanA primers comprises or consists of a forward primer of the sequence of SEQ ID NOs: 1 or 2, or a complement thereof, and a reverse primer of the sequence of SEQ ID NO: 3 or 4, or a complement thereof; the set of vanB primers comprises or consists of a forward primer of a sequence selected from the group consisting of SEQ ID NOs: 6, 7, 11 and 12, or a complement thereof and a reverse primer of a sequence selected from the group consisting of SEQ ID NO: 8, 9, 13 and 14, or a complement thereof; and wherein the one or more detectable vanA probe comprises or consists of the sequence of SEQ ID NO: 5, or a complement thereof, and the one or more detectable vanB probe comprises or consists of the sequence of SEQ ID NOs: 10 or 15, or a complement thereof.

2. The method of claim 1, wherein:
the hybridizing step comprises contacting the amplification product with a detectable probe that is labeled with a donor fluorescent moiety and a corresponding acceptor fluorescent moiety; and
the detecting step comprises detecting the presence or absence of fluorescence resonance energy transfer (FRET) between the donor fluorescent moiety and the acceptor fluorescent moiety of the probe, wherein the presence or absence of fluorescence FRET is indicative of the presence or absence of in the sample.

3. The method of claim 2, wherein said amplifying step employs a polymerase enzyme having 5′ to 3′ nuclease activity.

4. The method of claim 2, wherein the donor fluorescent moiety and the corresponding acceptor fluorescent moiety are within no more than 8 nucleotides of each other on the probe.

5. The method of claim 2, wherein the acceptor fluorescent moiety is a quencher.

6. The method of claim 1, wherein the set of vanA primers comprises or consists of a forward primer of SEQ ID NO: 2, and a reverse primer of SEQ ID NO: 4 and the set of vanB primers comprises or consists of a forward primer of SEQ ID NO: 7 and a reverse primer of SEQ ID NO: 9, and the one or more detectable vanB probe comprises or consists of SEQ ID NO: 10.

7. A primer and probe set for amplification and detection of vanA gene target sequence comprising:
forward and reverse primers comprising or consisting of the nucleotide sequences of SEQ ID NOs: 1, 2, 3, and 4, or a complement thereof, and a detectable probe for detection of vanA amplification product comprising or consisting of the nucleic acid sequence of SEQ ID NO: 5, or a complement thereof.

8. The primer and probe set of claim 7 wherein the detectable probe comprises a donor fluorescent moiety and a corresponding acceptor fluorescent moiety.

9. The primer and probe set of claim 8 wherein the acceptor fluorescent moiety is a quencher.

10. A primer and probe set for amplification and detection of vanB gene target sequence comprising:
forward and reverse primers comprising or consisting of the nucleic acid sequences of SEQ ID NOs: 6, 7, 8, 9, 11, 12, 13 and 14 or a complement thereof, and a detectable probe for detection of the vanB amplification product comprising or consisting of the nucleic acid sequence of SEQ ID NOs: 10 or 15, or a complement thereof.

11. The primer and probe set of claim 10 wherein the detectable probe comprises a donor fluorescent moiety and a corresponding acceptor fluorescent moiety.

12. The primer and probe set of claim 11 wherein the acceptor fluorescent moiety is a quencher.

13. A kit for detecting a nucleic acid of vanA-containing Staphylococcus aureus comprising the primer and probe set of claim 7 and further comprising nucleoside triphosphates, nucleic acid polymerase, and buffers necessary for the function of the nucleic acid polymerase.

14. The kit of claim 13, wherein the detectable probe comprises a donor fluorescent moiety and a corresponding acceptor fluorescent moiety.

15. The kit of claim 14, wherein the acceptor fluorescent moiety is a quencher.

16. A method for detection of Staphylococcus aureus (S. aureus) having vancomycin resistance (VRSA), methicillin resistance (MRSA) or both VRSA and MRSA, the method comprising:
performing an amplifying step including contacting the sample with: a set of vanA forward and reverse primers and a set of vanB forward and reverse primers to produce an amplification product if the vanA gene and/or vanB gene is present in the sample; a set of forward and reverse primers to produce an amplification product of the S. aureus capsular polysaccharide enzyme (CPE) if S. aureus is present in the sample; a set of forward and reverse primers to produce an amplification product of the right extremity junction of the Staphylococcal Chromosomal Cassette mec (SCCmec) mobile genetic element if SCCmec is present in the sample; and a set of mecA forward and reverse primers and a set of mecC forward and reverse primer to produce an amplification product if the mecA and/or mecC gene is present in the sample;
performing a hybridizing step including contacting the amplification products with one or more detectable vanA probes, one or more detectable vanB probes, one or more detectable CPE probes, one or more detectable SCCmec probes, one or more detectable mecA probes, and one or more detectable mecC probes; and
detecting the presence or absence of the amplification products, wherein the presence of the amplification product of the vanA gene and/or the vanB gene and the CPE gene is indicative of the presence of VRSA in the sample and wherein the presence of the amplification product of the SCCmec element, the mecA gene and/or the mecC gene and the CPE gene is indicative of the presence of MRSA in the sample.

17. The method of claim 16, wherein
the set of vanA forward and reverse primers for amplification of the vanA gene includes nucleic acid sequences of SEQ ID NOs: 1, 2, 3, and 4, or a complement thereof, and the one or more detectable probe for detection of the vanA amplification product includes the nucleic acid sequence of SEQ ID NO: 5, or a complement thereof;
the set of vanB forward and reverse primers for amplification of the vanB gene include nucleic acid sequences of SEQ ID NOs: 6, 7, 8, 9, 11, 12, 13 and 14 or a complement thereof, and the one or more detectable probe for detection of the vanB amplification product includes the nucleic acid sequence of SEQ ID NOs: 10 and 15, or a complement thereof;
the set of S. aureus CPE forward and reverse primers for amplification of the S. aureus CPE gene include nucleic acid sequences of SEQ ID NOs: 16 and 17 or a complement thereof, and the one or more detectable probe for detection of the S. aureus CPE amplification product includes the nucleic acid sequence of SEQ ID NO: 18, or a complement thereof;
the set of SCCmec forward and reverse primers for amplification of the SCCmec element include nucleic acid sequences of SEQ ID NOs: 19, 20, 21, 22, 23 and 24, or a complement thereof, and the one or more detectable probe for detection of the SCCmec amplification product include nucleic acid sequence os SEQ ID NOs: 25, or a complment thereof;
the set of mecA forward and reverse primers for amplification of the mecA gene includes nucleic acid sequences of SEQ ID NOs: 26 and 27, or a complement thereof, and the one or more detectable probe for detection of the mecA amplification product includes the nucleic acid sequence of SEQ ID NO: 28, or a complement thereof; and
the set of mecC forward and reverse primers for amplification of the mecC gene includes nucleic acid sequences of SEQ ID NOs: 29 and 30, or a complement thereof, and the one or more detectable probe for detection of the mecC amplification product includes the nucleic acid sequence of SEQ ID NO: 31, or a complement thereof.


SEQUENCE LISTING (31 sequences)

SEQ ID NO: 1 - DNA, 27 bp, synthetic construct
  misc_feature 1..27: note = vanA forward primer
  source 1..27: mol_type = other DNA
ggaattacgaaatctggtgtatggaaa

SEQ ID NO: 2 - DNA, 27 bp, synthetic construct
  misc_feature 1..27: note = vanA forward primer SEGP3128_TBB
  modified_base 27: mod_base = OTHER; note = t-butylbenzyl-dA
  source 1..27: mol_type = other DNA
ggaattacgaaatctggtgtatggaaa

SEQ ID NO: 3 - DNA, 27 bp, synthetic construct
  misc_feature 1..27: note = vanA reverse primer
  source 1..27: mol_type = other DNA
ggttctttttaacaagtaatccgtgca

SEQ ID NO: 4 - DNA, 27 bp, synthetic construct
  misc_feature 1..27: note = vanA reverse primer SEGP3129_TBB
  modified_base 27: mod_base = OTHER; note = t-butylbenzyl-dA
  source 1..27: mol_type = other DNA
ggttctttttaacaagtaatccgtgca

SEQ ID NO: 5 - DNA, 36 bp, synthetic construct
  misc_feature 1..36: note = vanA probe SEGP3130_vanA_ATTO
  misc_feature 1..36: note = 5` ATTO425N dye
  misc_feature 1..36: note = 3` phosphate
  misc_feature 9..10: note = BHQ2 Quencher
  source 1..36: mol_type = other DNA
tgggaaaacgacaattgctattcagctgtactctcg

SEQ ID NO: 6 - DNA, 24 bp, synthetic construct
  misc_feature 1..24: note = vanB forward primer
  source 1..24: mol_type = other DNA
gcaggtgtgggattgctttatcaa

SEQ ID NO: 7 - DNA, 24 bp, synthetic construct
  misc_feature 1..24: note = vanB forward primer SEGP3124_TBB
  modified_base 24: mod_base = OTHER; note = t-butylbenzyl-dA
  source 1..24: mol_type = other DNA
gcaggtgtgggattgctttatcaa

SEQ ID NO: 8 - DNA, 25 bp, synthetic construct
  misc_feature 1..25: note = vanB reverse primer
  source 1..25: mol_type = other DNA
cattttattcgcactgtcccgctaa

SEQ ID NO: 9 - DNA, 25 bp, synthetic construct
  misc_feature 1..25: note = vanB reverse primer SEGP3135_TBB
  modified_base 25: mod_base = OTHER; note = t-butylbenzyl-dA
  source 1..25: mol_type = other DNA
cattttattcgcactgtcccgctaa

SEQ ID NO: 10 - DNA, 37 bp, synthetic construct
  misc_feature 1..37: note = vanB probe SEGP3136_vanB_ATTO
  misc_feature 1..37: note = 5` ATTO425N dye
  misc_feature 1..37: note = 3` phosphate
  misc_feature 9..10: note = BHQ2 Quencher
  source 1..37: mol_type = other DNA
tgattgcgataatggttgtattcagccttttatgcgc

SEQ ID NO: 11 - DNA, 27 bp, synthetic construct
  misc_feature 1..27: note = vanB forward primer
  source 1..27: mol_type = other DNA
gcacagcaatttgtgtcttatttcaga

SEQ ID NO: 12 - DNA, 27 bp, synthetic construct
  misc_feature 1..27: note = vanB forward primer SEGP3137_TBB
  modified_base 27: mod_base = OTHER; note = t-butylbenzyl-dA
  source 1..27: mol_type = other DNA
gcacagcaatttgtgtcttatttcaga

SEQ ID NO: 13 - DNA, 29 bp, synthetic construct
  misc_feature 1..29: note = vanB reverse primer
  source 1..29: mol_type = other DNA
ctcaaaggattggttattgtagtgaaaca

SEQ ID NO: 14 - DNA, 29 bp, synthetic construct
  misc_feature 1..29: note = vanB reverse primer SEGP3138_TBB
  modified_base 29: mod_base = OTHER; note = t-butylbenzyl-dA
  source 1..29: mol_type = other DNA
ctcaaaggattggttattgtagtgaaaca

SEQ ID NO: 15 - DNA, 36 bp, synthetic construct
  misc_feature 1..36: note = vanB probe SEGP3139_vanB_ATTO
  misc_feature 1..36: note = 5` ATTO425N dye
  misc_feature 1..36: note = 3` phosphate
  misc_feature 9..10: note = BHQ2 Quencher
  source 1..36: mol_type = other DNA
tcagccattggtggaactgattcagaatagcgatag

SEQ ID NO: 16 - DNA, 27 bp, synthetic construct
  misc_feature 1..27: note = CPE forward primer JJ152BBC
  modified_base 27: mod_base = OTHER; note = t-butylbenzyl-dC
  source 1..27: mol_type = other DNA
aagataagcttattgaacaaggacatc

SEQ ID NO: 17 - DNA, 23 bp, synthetic construct
  misc_feature 1..23: note = CPE reverse primer JJ157BBC
  modified_base 23: mod_base = OTHER; note = t-butylbenzyl-dC
  source 1..23: mol_type = other DNA
cttgaggtgaattgttgtgaacc

SEQ ID NO: 18 - DNA, 30 bp, synthetic construct
  misc_feature 1..30: note = CPE probe SAP8
  misc_feature 1..30: note = 5` fluorescein dye
  misc_feature 1..30: note = 3` 3IABkFQ Quencher
  misc_feature 8..9: note = ZEN Quencher
  source 1..30: mol_type = other DNA
ttaggaatcaattatggaagtcgacctcgt

SEQ ID NO: 19 - DNA, 24 bp, synthetic construct
  misc_feature 1..24: note = orfX forward primer ORFX_F1
  modified_base 24: mod_base = OTHER; note = t-butylbenzyl-dA
  source 1..24: mol_type = other DNA
cagcaaaatgacattcccacatca

SEQ ID NO: 20 - DNA, 24 bp, synthetic construct
  misc_feature 1..24: note = orfX forward primer ORFX_F2
  modified_base 24: mod_base = OTHER; note = t-butylbenzyl-dA
  source 1..24: mol_type = other DNA
cagcaaaatgacatttccacacca

SEQ ID NO: 21 - DNA, 36 bp, synthetic construct
  misc_feature 1..36: note = orfX reverse primer ORFX_R-2
  modified_base 36: mod_base = OTHER; note = t-butylbenzyl-dA
  source 1..36: mol_type = other DNA
cctccacatctcattaaatttttaaattatacacaa

SEQ ID NO: 22 - DNA, 37 bp, synthetic construct
  misc_feature 1..37: note = orfX reverse primer ORFX_R-4
  modified_base 37: mod_base = OTHER; note = t-butylbenzyl-dA
  source 1..37: mol_type = other DNA
ttcttcaaagatttgagctaatttaataattttctca

SEQ ID NO: 23 - DNA, 28 bp, synthetic construct
  misc_feature 1..28: note = orfX reverse primer ORFX_R-5
  modified_base 28: mod_base = OTHER; note = t-butylbenzyl-dA
  source 1..28: mol_type = other DNA
ggaggctaactatgtcaaaaatcatgaa

SEQ ID NO: 24 - DNA, 28 bp, synthetic construct
  misc_feature 1..28: note = orfX reverse primer ORFX_R-1-2N1
  modified_base 28: mod_base = OTHER; note = t-butylbenzyl-dA
  source 1..28: mol_type = other DNA
gaggttaaaaaaaccgcatcatttgtga

SEQ ID NO: 25 - DNA, 39 bp, synthetic construct
  misc_feature 1..39: note = orfX probe ORFX_A_SIMA
  misc_feature 1..39: note = 5` HEX dye
  misc_feature 1..39: note = 3` 3IABkFQ Quencher
  misc_feature 7..8: note = ZEN Quencher
  source 1..39: mol_type = other DNA
gatgcgggttgtgttaattgaacaagtgtatagagcatt

SEQ ID NO: 26 - DNA, 29 bp, synthetic construct
  misc_feature 1..29: note = mecA forward primer MECA_F1006
  modified_base 29: mod_base = OTHER; note = t-butylbenzyl-dC
  source 1..29: mol_type = other DNA
atgatgcagttattggtaaaaagggactc

SEQ ID NO: 27 - DNA, 30 bp, synthetic construct
  misc_feature 1..30: note = mecA reverse primer MECA_R1077
  modified_base 30: mod_base = OTHER; note = t-butylbenzyl-dC
  source 1..30: mol_type = other DNA
tgtatgtgcgattgtattgctattatcgtc

SEQ ID NO: 28 - DNA, 35 bp, synthetic construct
  misc_feature 1..35: note = mecA probe MECA_P2_TAO9
  misc_feature 1..35: note = 5` LCR640 dye
  misc_feature 1..35: note = 3` IBRQ Blocker
  misc_feature 9..10: note = TAO Quencher
  source 1..35: mol_type = other DNA
ctccaacatgaagatggctatcgtgtcacaatcgt

SEQ ID NO: 29 - DNA, 29 bp, synthetic construct
  misc_feature 1..29: note = mecC forward primer MECC_F1_TBB
  modified_base 29: mod_base = OTHER; note = t-butylbenzyl-dA
  source 1..29: mol_type = other DNA
acggcaatatcgatttaaagcaagcaata

SEQ ID NO: 30 - DNA, 31 bp, synthetic construct
  misc_feature 1..31: note = mecC reverse primer MECC_R1_TBB
  modified_base 31: mod_base = OTHER; note = t-butylbenzyl-dA
  source 1..31: mol_type = other DNA
tgctttataaaagggataatcactcgggata

SEQ ID NO: 31 - DNA, 33 bp, synthetic construct
  misc_feature 1..33: note = mecC probe MECCP1_LCR9.1
  misc_feature 1..33: note = 5` LCR640 dye
  misc_feature 1..33: note = 3` IBRQ Blocker
  misc_feature 10..11: note = TAO Quencher
  source 1..33: mol_type = other DNA
tgagcaaggtatgcaagatttgggaatcggtga